(19) United States
(12) Patent Application Publication
(10) Pub. No.: US 2006/0265040 A1
(43) Pub. Date: Nov. 23, 2006

(54) Stent delivery and retention apparatus

(75) Inventor: Robert J. Murray, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/130,725

(22) Filed: May 17, 2005

(51) Int. Cl. A61F 2/06

(52) U.S. Cl. 623/1.11

(57) ABSTRACT

A stent delivery system comprises an inner member and a collapsible balloon mounted in a collapsed state thereon. A compressible stent is mounted around the collapsible balloon. At least a first thermoplastic or elastomeric member is positioned between the collapsible balloon and the stent to increase the retention force between the collapsible balloon and the stent.

### FIELD OF THE INVENTION

This invention relates generally to an intravascular stent deployment apparatus, and more particularly to a stent delivery apparatus including an adhesive member for increasing the stent retention force between the stent and a balloon upon which the stent is compressed.

### BACKGROUND OF THE INVENTION

In a typical percutaneous transluminal coronary angioplasty (PTCA) procedure, a guiding catheter is percutaneously introduced into the cardiovascular system of a patient. The guide catheter is advanced through a vessel until the distal end thereof is at desired location in the vasculature. A guide wire and a dilatation catheter having a balloon on the distal end thereof are introduced into the guiding catheter with the guidewire sliding through the dilatation catheter. The guide wire is first advanced out of the guiding catheter into the patient's coronary vasculature, and the dilatation catheter is advanced over the previously advanced guide wire until the dilatation balloon is properly positioned across the lesion. Once in position, the flexible, expandable, preformed balloon is inflated to a predetermined size with a liquid or gas at relatively high pressures (e.g. about ten to twelve atmospheres) to radially compress the arthrosclerotic plaque in the lesion against the inside of the artery wall and thereby dilate the lumen of the artery. The balloon is then deflated to a small profile so that the dilatation catheter may be withdrawn from the patient's vasculature and blood flow resumed through the dilated artery.

In angioplasty procedures of the kind described above, there may occur a restenosis of the artery; i.e., a re-narrowing of the treated coronary artery which is related to the development of neo-intinmal hyperplasia that occurs within the artery after it has been treated as described above. In a sense, restenosis is scar tissue that forms in response to mechanical intervention within a vascular structure. To prevent restenosis and strengthen the area, an intravascular prosthesis generally referred to as a stent can be implanted for maintaining vascular patency inside the artery at the lesion. The stent is mounted in a compressed state around a deflated balloon, and the balloon/stent assembly maneuvered through a patient's vasculature to the site of a target lesion. The stent is then expanded to a larger diameter for placement or implantation in the vasculature. The stent effectively overcomes the natural tendency of the vessel walls of some patients to close back down, thereby maintaining a normal flow of blood through the vessel that would not be possible if the stent was not in place.

A known expandable stent, which is delivered on a balloon catheter, may be considered to be a stainless steel cylinder having a number of slits in its circumference resulting in a mesh when expanded. The stainless steel cylinder is compressed onto the outside of a non-expanded balloon catheter which includes stent retainer rings at each end of the stent to help maintain the stent on the balloon. Unfortunately, the limited amount of securment between the stent and the balloon is not always adequate to insure that the stent will properly stay in place while advancing the stent to and through a target lesion. Additionally, the outer surface of the delivery device is uneven because the stent generally extends radially outward beyond the balloon. Thus, the stent may contact a vessel wall and be displaced while the catheter negotiates a narrow vessel. Furthermore, during a coronary intervention, the physician may have difficulty crossing the target lesion. In such cases, it may be necessary to pull the stent delivery system back into the guide catheter. Such procedures can cause premature displacement of the stent resulting in serious risk to the patient.

For example, the guide catheter is generally inserted through the abdominal aorta to a point just beyond the ostium, the location from which the right coronary artery and the left main artery diverge from the aorta. Blockages or lesions are typically present in smaller coronary vessels, and medical practitioners may sometimes predialate the target area as, for example, by balloon angioplasty. Sometimes, however, predialation is not performed, and doctors proceed directly to a primary stenting procedure. In such cases, there are occasions when the balloon/stent catheter cannot be properly positioned within the target area due to the constriction of the vessel and must be retracted back into the guide catheter. Even when predialation is performed, vascular spasms and/or a reclosure of the vessel may occur rendering it difficult to properly align the balloon/stent assembly and likewise requiring retraction into the guide catheter. In addition, the lesion may be heavily calcified requiring a high insertion pressure. In either case, unwanted displacement of the compressed stent may occur.

It should therefore be appreciated that it would be desirable to provide a low profile stent delivery and deployment apparatus that provides an increased interference or retention force between the compressed stent and the balloon.

### SUMMARY OF THE INVENTION

According to an aspect of the invention, there is provided a stent delivery system comprising an inner member and a collapsible balloon mounted in a collapsed state thereon. A compressible stent is mounted around the collapsible balloon. At least a first thermoplastic member is positioned between the collapsible balloon and the stent to increase the retention force between the collapsible balloon and the stent.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and;

FIG. 1 is a longitudinal view of a stent and balloon assembly in accordance with the present invention;

FIG. 2 is a longitudinal view of the stent and balloon assembly shown in FIG. 1 with a portion of the stent removed to expose adhesive or tape strips for increasing the balloon/stent retention force;

FIG. 3 and FIG. 4 are cross-sectional views of the balloon/stent assembly shown in FIG. 2 taken along line 3-3 and line 4-4 respectively;

FIG. 5 is an enlarged view of a section of FIG. 4;

FIG. 6 is a longitudinal view of a second embodiment of the present invention illustrating the use of an elastomeric material wrapped in a spiral configuration around the balloon for increasing the balloon/stent retention force; and

FIG. 7 is a longitudinal view of a third embodiment illustrating the use of an elastomeric sleeve between the balloon and stent and having longitudinal slots therein.

### DETAILED DESCRIPTION OF THE EXEMPLARY EMBODIMENTS

The following description is exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

FIG. 1 is a longitudinal, cross-sectional view of a balloon/stent assembly embodying the principles of the present invention. The balloon/stent assembly shown generally at 20 comprises a stent 22, an inner member or wire lumen 24 having a distal end 26 and a proximal end 28, and distal and proximal radiopaque marker bands 30 and 32 respectively which are positioned on inner member or wire lumen 24 near the distal and proximal ends of stent 22. Stent 22 may be of any form or configuration suitable for the intended purpose, and may comprise one or more stent segments depending on the size and configuration of the vessel to be treated. It will be recognized by those skilled in the art that inner member or guide lumen 24 is configured for the insertion of a conventional guide wire (not shown) which will enable the balloon/stent assembly to be guided and positioned at a target location in the vessel to be treated.

Any conventional or modified balloon catheter device may be used such as a PTCA balloon catheter. An expandable balloon portion 34 is mounted on inner member 24 in a compressed or collapsed state beneath stent 22 and extends beyond the proximal and distal ends of stent 22. Balloon 34 is generally made of a soft delicate material such as polyethylene, polyethylene terathalate, nylon or the like. The length and the diameter of the balloon may be selected to accommodate the particular configuration of the stent to be deployed. Stent 22 may be constructed of any implantable material having good mechanical strength, such as implantable quality stainless steel. The outside of the stent may be selectively plated with platinum or other implantable radiopaque substance to provide visibility during fluoroscopy. The cross-sectional shape of the finished stent 22 may be circular, ellipsoidal, rectangular, hexagonal, square, or any other desired shape, although a circular or ellipsoidal cross-section is preferable. The length and width of stent 22 is generally determined to a large degree by the size of the vessel into which the stent will be deployed. Stent 22 must be of sufficient length to maintain its axial orientation without shifting under the hydraulics of blood flow, long enough to extend across a significant portion of the target area, and at the same time not be unnecessarily long so as to result in the introduction of an unnecessarily large amount of material into the vessel.

After stent selection, the stent 22 is compressed upon the outside of balloon 34. An inner sheath (not shown) is placed over each end of balloon 34, and an exterior sheath (also not shown) is placed over the ends of the interior sheath so as to cover stent 22 and overlap with the interior sheaths. The assembly is then pressurized by introducing air or an inert gas such as nitrogen through the lumen 24 into the interior of balloon 34 so as to expand the balloon within the sheaths. The assembly is then exposed to an elevated temperature while maintaining pressurization of the balloon. Following heating, the balloon/stent assembly is allowed to cool within the sheaths, and this cooling sets the shape of balloon 34. The sheaths may then be removed. This process is described in detail in U.S. Pat. No. 5,836,965 entitled “Stent Delivery and Deployment Method” issued Nov. 17, 1998, the teachings of which are hereby incorporated by reference.

Marker bands 30 and 32, which may be viewed through fluoroscopy, assist in positioning the assembly. When the assembly is properly located across a lesion, the balloon may be inflated in a conventional manner. This results in the general uniform, symmetrical expansion of the stent and balloon. The amount of inflation and thus the amount of expansion of the stent may be varied as dictated by the lesion itself.

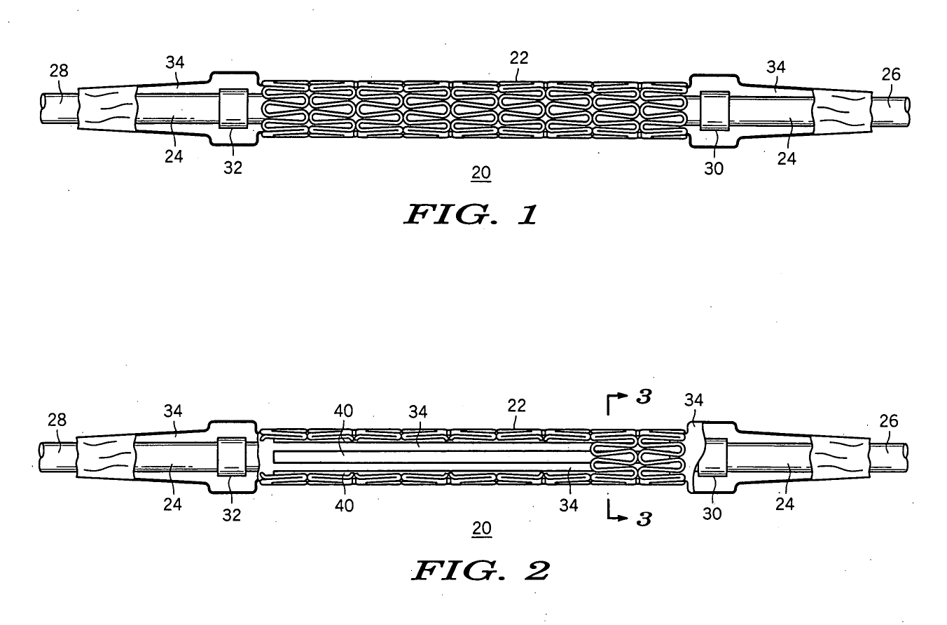

FIG. 2 is a longitudinal view of the balloon/stent assembly shown in FIG. 1 with a portion of the stent removed so as to illustrate the use of a thermoplastic material such as strips of compressible and formable tape or adhesive or otherwise bondable strips for increasing the force of retention between the balloon and stent. Referring to FIG. 2, a plurality of strips 40 is longitudinally secured to balloon 34 around its periphery. It should be clear that the number of longitudinal strips utilized might vary with different balloon/stent assemblies or applications. For example, in some cases only a single strip of tape 40 is needed. Furthermore, a retention strip or strips may be positioned circumferentially around the balloon, provided that the strip or strips do not cross any of the balloon folds. If desired, strips 40 could be made of the same material as balloon 34 and created at the time of balloon manufacture.

Tape 40 may be made of any soft, foam-like material which has high compressibility and formability. For example, tape strips 40 may be made of a non-woven Polyurethane foam tape, such as the type which is available from 3M. Each strip of tape may have a thickness of, for example, 0.6 millimeters and a width of, for example, 2-4 millimeters. A tape strip 40 is preferably applied at the edge of a balloon fold or folds. Stent 22 is then crimped over tape strips 40 and balloon 34 and processed as described above. During the heating process, the foam tape softens allowing the material to fill voids in stent 22.

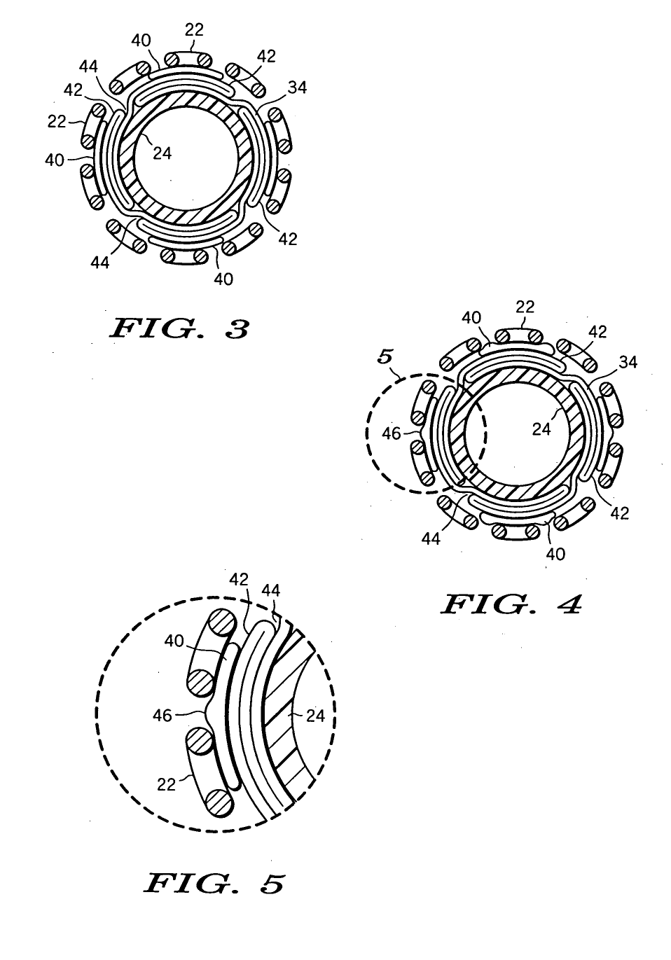

FIG. 3 is a cross-sectional view of the balloon/stent assembly shown in FIG. 2 taken along line 3-3. As can be seen, balloon 34 is collapsed upon inner member 24 such that a plurality of folds (in this case four) 42 is produced. Each fold 42 has a longitudinal edge 44. The wings or folds 42 of balloon 34 may be formed by pulling the balloon catheter through a forming tool having a generally cylindrical cross-section and defining a terminal opening configured to produce the desired number of wings or folds in the balloon. For example, the terminal opening may include four slits extending radially outward from the end of the forming tool, the number of slits depending upon the number of folds to be produced. As the balloon catheter is pulled through the forming tool, the balloon is pushed through the terminal opening and exits having, for example, four separate flutes. The balloon catheter bearing the fluted balloon portion is then pulled into a sheath, preferably a two-part sheath made of Teflon or other suitable material so that the flutes fold and wrap around the catheter in a clockwise direction to form a generally spiral configuration. The sheath/balloon catheter assembly is then heated, preferably by placing the assembly in an oven, to form a crease in substantially the length of each of the folded flutes. Following heating, balloon 34 retains the creases formed in the wings to define a generally symmetrical, cylindrical cross-section as can be seen in FIG. 3.

Referring now to FIG. 3, it can be seen that four folds 42 have been formed in balloon 34 and have been wrapped around inner member 24. Each fold 42 has an edge 44. Strips 40 are adhesively coupled to the balloon proximate the edge of the fold 44. A variety of adhesives are suitable for this purpose. For example, ultra-violet cure and many epoxy and cyanoacrylate type adhesives are suitable. Stent 22 is then crimped or compressed over strips 40 as is shown in FIG. 3. The assembly is then heated as described above, and during the heating process, adhesive foam strips 40 soften and at least partially fill the voids in stent 22 as is shown at 46 in FIG. 4 and more clearly in the enlarged view of FIG. 5.

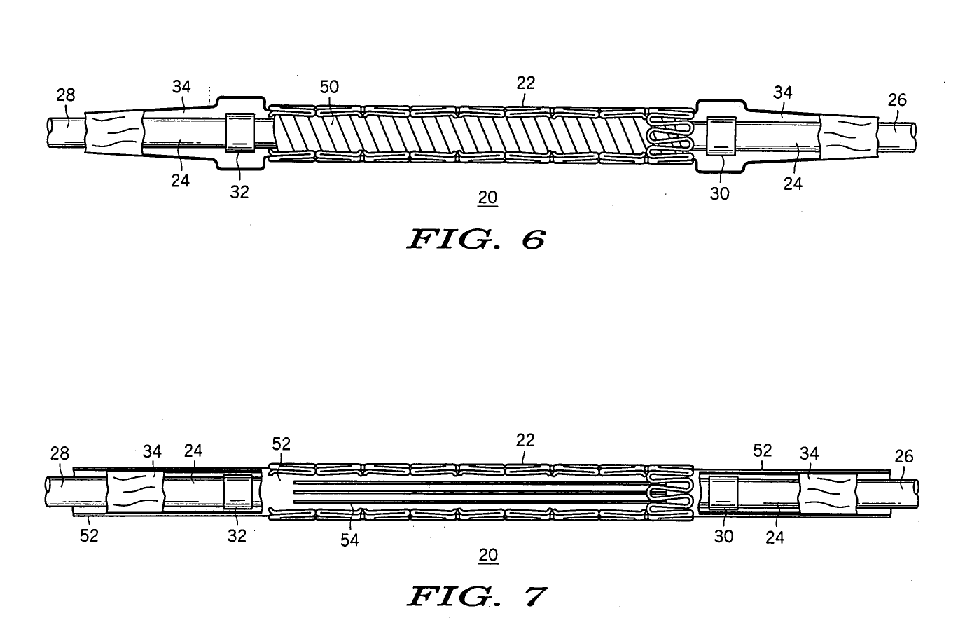

FIG. 6 illustrates an alternate embodiment of the present invention. In this case, an elastomeric material 50 (e.g. polyurethane, silicone rubber, etc.) is wrapped in a spiral fashion around balloon 34 at least in the region under stent 22. The tape may be wound in a tight spiral as is the case shown in FIG. 6 or in a loose spiral such that there are exposed spaces between adjacent edges of the tape in the spiral.

FIG. 7 illustrates a still further embodiment of the present invention. In this embodiment, a tube or sleeve of elastomeric material 52 is slipped over collapsible balloon 34. Tube or sleeve 52 may cover only that region beneath stent 22 or may in fact extend beyond the proximal and distal ends of stent 22. Tube or sleeve 52 may be made of any suitable elastomeric material such as polyurethane. The use of a solid sleeve could require some mechanism for pressure relief when balloon 34 is inflated. This may be accomplished by providing longitudinal slits 54 in the sleeve. To facilitate the mounting of tube or sleeve 52 onto collapsible balloon 34, the tube may be cut into a spiral shape and wound onto collapsible balloon 34. This would result in an appearance similar to that shown in FIG. 6.

Thus, there has been provided an intravascular support device wherein a soft, foam-like material having a high compressibility and formability when heated is inserted between a collapsible balloon and a stent compressed thereon. The material may comprise strips of, for example, a Polyurethane foam tape which may be adhesively attached to folds in the collapsed balloon. Alternatively, a sleeve of elastomeric material may be slipped over the collapsed balloon prior to compression of the stent thereon. In either case, the retention force between the stent and the collapsed balloon is enhanced thereby preventing unwanted displacement of the stent on the balloon during a stent deployment procedure.

In the foregoing specification, the invention has been described with reference to specific embodiments. It should be appreciated, however, that various modifications and changes might be made without departing from the scope of the invention as set forth in the appended claims. Accordingly, the specification and figures should be regarded as illustrative rather than restrictive, and all such modifications are intended to be included within the scope of the present invention.

1. A stent delivery system, comprising:
an inner member;
a collapsible balloon mounted in a substantially collapsed state on said inner member;
at least a first thermoplastic member positioned on said collapsed balloon; and
a compressible stent mounted in a substantially compressed state around said collapsible balloon and said at least a first thermoplastic member.

2. A stent delivery system according to claim 1 wherein said at least a first thermoplastic member is a strip of compressible and formable material.

3. A stent delivery system according to claim 2 wherein said strip is positioned substantially longitudinally on said collapsible balloon.

4. A stent delivery system according to claim 2 wherein said strip is positioned substantially circumferentially around said collapsible balloon.

5. A stent delivery system according to claim 3 wherein said at least a first thermoplastic member comprises a plurality of compressible strips positioned substantially longitudinally along said collapsible balloon.

6. A stent delivery system according to claim 5 wherein each of said plurality of compressible strips is bonded to said collapsible balloon.

7. A stent delivery system according to claim 6 wherein said plurality of compressible strips is adhesively fixed to said collapsible balloon.

8. A stent delivery system according to claim 6 wherein said plurality of compressible strips is formed when said collapsible balloon is formed.

9. A stent delivery system according to claim 7 wherein said collapsible balloon comprises a plurality of folds around said inner member, each fold having an edge, and wherein each one of said plurality of compressible strips is positioned substantially along one of said edges.

10. A stent delivery system according to claim 5 wherein said plurality of compressible strips is made of the same material as said collapsible balloon.

11. A stent delivery system according to claim 5 wherein said compressible strips contain elastomer, such as polyurethane or silicone.

12. A stent delivery system according to claim 5 wherein each of said plurality of compressible strips has a width of approximately 1-4 millimeters and a thickness less than 1 mm.

13. A stent delivery system according to claim 2 wherein said strip is an elastomeric strip wrapped in a substantially spiral fashion around at least a portion of said collapsible balloon.

14. A stent delivery system according to claim 1 wherein said at least a first thermoplastic member is a sleeve of elastomeric material positioned around at least a portion of said collapsible balloon.

15. A stent delivery system according to claim 14 wherein said elastomeric material contains polyurethane.

16. A stent delivery system according to claim 14 wherein said sleeve has a plurality of longitudinal slots therein.

17. A stent delivery system according to claim 14 wherein said sleeve is cut into a spiral configuration.

18. A stent delivery system, comprising:
an inner member;
a collapsible balloon mounted in a substantially collapsed state on said inner member;
a compressible stent mounted in a substantially compressed state around said collapsible balloon; and
at least a first thermoplastic member positioned between said collapsible balloon and said stent to increase retention force between said collapsible balloon and said stent.

19. A stent delivery system according to claim 18 wherein said at least a first thermoplastic member is a strip of compressible and formable material.

20. A stent delivery system according to claim 19 wherein said strip is positioned substantially longitudinally on said collapsible balloon.

21. A stent delivery system according to claim 20 wherein said at least a first thermoplastic member comprises a plurality of compressible and formable strips positioned substantially longitudinally along said collapsible balloon.

22. A stent delivery system according to claim 21 wherein said plurality of compressible and formable strips is bonded to said collapsible balloon.

23. A stent delivery system according to claim 22 wherein said plurality of compressible and formable strips is adhesively fixed to said collapsible balloon.

24. A stent delivery system according to claim 21 wherein said plurality of compressible and formable strips is formed when said collapsible balloon is formed.

25. A stent delivery system according to claim 22 wherein said collapsible balloon comprises a plurality of folds around said inner member, each fold having an edge, and wherein each one of said plurality of compressible and formable strips is positioned substantially along one of said edges.

26. A stent delivery system according to claim 24 wherein said plurality of compressible and formable strips is made of the same material as said collapsible balloon.

27. A stent delivery system according to claim 19 wherein said strip is an elastomeric strip wrapped in a substantially spiral fashion around at least a portion of said collapsible balloon.

28. A stent delivery system according to claim 18 wherein said at least a first thermoplastic member is a sleeve of elastomeric material positioned around at least a portion of said collapsible balloon.

29. A stent delivery system according to claim 28 wherein said sleeve has a plurality of longitudinal slots therein.

30. A stent delivery system according to claim 28 wherein said sleeve is cut into a spiral configuration prior to placement on said collapsible balloon.
